(19) United States
(12) Patent Application Publication
(10) Pub. No.: US 2024/0428422 A1
(43) Pub. Date: Dec. 26, 2024

(54) A SYSTEM AND METHOD FOR QUALITY ASSURANCE OF MEDICAL IMAGE SEGMENTATION

(71) Applicant: Mirada Medical Limited, Oxord (GB)

(72) Inventors: Georgia-Valerie IONESCU, Oxford (GB); Ana OLIVEIRA, Oxford (GB); Padraig LOONEY, Oxford (GB); Marcel VAN HERK, Oxford (GB); Marianne AZNAR, Oxford (GB); Mark John GOODING, Oxford (GB); Djamal BOUKERROUI, Oxford (GB); Eliana Vasquez OSORIO, Oxford (GB)

(21) Appl. No.: 18/699,291

(22) PCT Filed: Sep. 28, 2022
(86) PCT No.: PCT/EP2022/077002

§ 371 Date: Apr. 7, 2024

(30) Foreign Application Priority Data
Oct. 7, 2021 (GB) 2114398.7
May 19, 2022 (GB) 2207358.9

(51) Int. Cl. G06T 7/12; A61N 5/10; G06T 7/00; G06T 11/20

(52) U.S. Cl. CPC G06T 7/12; G06T 7/0012; G06T 11/203; A61N 5/103; G06T 2207/10081; G06T 2207/10088; G06T 2207/10104; G06T 2207/10108; G06T 2207/20081; G06T 2207/20092; G06T 2207/20128; G06T 2207/30004; G06T 2207/30168; G06T 2210/41

(57) ABSTRACT

A method and system for reviewing contouring of medical images in a contouring system is described. The method comprising the steps of: providing at least one medical image showing structures to be contoured; generating a contour for a structure on the at least one medical image; determining if the generated contour conforms to guidelines for the structure being contoured; in response to the determining of conformity providing feedback about the quality of the contour; and continuing to generate the contour based on the provided feedback. A method and system for reviewing previously contoured medical images is also described.

### FIELD OF INVENTION

This invention relates to the fields of medical imaging and medical image processing, in particular to the review of contouring of medical images, and in particular in the field radiotherapy treatment planning.

### BACKGROUND OF INVENTION

In many scenarios, it is necessary for a clinician to outline anatomical structures on a medical image. For example, in radiotherapy treatment planning, organs, tumours and target volumes are typically delineated by a radiation oncologist on a medical image of the patient. The patient image is normally acquired with a Computed Tomography (CT) scan, although other imaging modalities, such as Magnetic Resonance Imaging (MRI), may be acquired. These images typically comprise a stack of 2D images of cross-sections of the patient, which together comprise a 3D volumetric image.

The medical images of the patient are outlined by a clinician to indicate which areas should be irradiated and which healthy tissues and organs should be avoided; over treatment may cause harm, while under-treatment can lead to the cancer recurring. Therefore, when preparing for treatment, organs-at-risk (OARs) and target volumes need to be delineated on a ‘planning’ image in order to spare healthy tissues while irradiating the tumour. The planning software can subsequently calculate a treatment plan that maximises radiation dose to the target volume and tumour, while minimising dose to the surrounding healthy tissues. This process of delineation is known as contouring, a term that may be used to indicate the process of delineation in either 2D or 3D to define the boundary of an anatomical structure. This contouring is time-consuming and skill-intensive, leading to variation in contours and consequent effect on the treatment. Similarly, the term auto-contouring may be used to indicate a contour produced automatically by a system.

Manual contouring of a patient case by a human operator is time consuming, and subject to variability in delineating the anatomical structures [1]. Such variability is due to intra-and inter-operator variation, and to variations between different institutions or departments. To reduce this variability, institutions and professional organisation produce contouring guidelines, such as [2], and atlases, such as [3]. The use of guidelines has been found to reduce variability between observers [4,5].

Guidelines take the form of a text description detailing how a structure should be drawn, while in this context, an atlas is a “gold-standard” set of contours drawn on an example patient image. Typically, guidelines and/or atlases are formed by consensus between experts.

While clinical teams are trained and educated in guidelines, over time variation can being to occur again where staff become complacent/forgetful [6], or guidelines get updated. This variation, and the risk of errors in contouring, can be mitigated through another senior clinician reviewing and revising the outlines, a process called peer-review. Therefore, peer-review is often suggested as a way to check contours, ensure their correctness, and remind staff of contouring guidelines. Peer-review of contours to ensure quality is strongly recommended [7,8,9].

However, this process is resource intensive, and in practice contours often are not checked against guidelines or peer-reviewed due to resource and time constraints. Therefore, there is need for a system to automatically check the adherence contouring to the guidelines.

Guidelines for contouring normally describe the correct contour for a structure being contoured with reference to;
anatomical boundaries between structures
other anatomical structures
or other anatomical features

These reference points will be referred to as the reference anatomy. The structure being contoured will be referred to as the target structure (although it may not be the target for radiotherapy—this term is sole being used to refer to the intended purpose of the user's contouring).

Guidelines may refer to contouring the target structure in a relative direction to the reference anatomy, for example; “Superiorly, the whole heart starts just inferior to the left pulmonary artery” [16], where the “left pulmonary artery” would be regarded as the reference anatomy, and the relative direction is “just inferior”.

Guidelines may refer to contouring the target structure at a distance from the reference anatomy, for example; “the lymph node CTV was defined as the area encompassed by a 7 mm margin around the applicable pelvic vessels (artery and vein)” [17], where “the applicable pelvic vessels” are the reference anatomy (further defined in the guidelines), and the distance is the 7 mm expansion margin. The “CTV” is a region known as a clinical target volume—a region where tumour cell is anticipated as being. Such regions are often expansions around a visible structure to account for non-visible tumour spread.

Guidelines may refer to contouring the target structure by the inclusion or exclusion of the reference anatomy, for example; “The CTV was modified to exclude bones and muscles” [17], where the “bones and muscles” are reference anatomy from exclusion of the contour of the “CTV” which is the target structure.

Several technical solutions have been proposed to assess the quality of the contours, with automated methods used to identify errors. Various techniques used to assess target and organ-at-risk contours have been published:

One class of approaches evaluates the patient contours with respect to the distribution of parameters of previously contoured cases [10]. However, such an approach only flags outliers with respect to a distribution and does not provide quality assurance with respect to contouring guideline. While the failure mode could be conveyed to the user, the user will still be required to interpret any contour with respect to the guideline. Furthermore, the system will not detect contours in violation of a guideline if they are with an expected patient distribution. For example, by comparing the volume of the heart with previously contoured heart-volumes we could infer that an abnormal big contour might be wrong and consequently need reviewing. Yet, the patient may have a big heart with respect to the population, and the contour may be correct. Conversely, if the heart had been incorrectly contoured too small, the contour may have an apparently “acceptable” volume within the normal distribution. Such a system does not relate the failure mode to the published contouring guidelines.

More complex machine learning approaches have also been applied to group feature to determine potential erroneous contours [11]. However, such approaches still fall into the category of detecting abnormal contours with respect to a population distribution, and do not relate the flagging of the case for review back to guidelines, as would occur in peer-review performed by a human.

Another class of approach is comparison of contours to automatically generated contours (auto-contours) [12]. In this approach the assumption is that the auto-contour is acceptable, thus deviations from it can be flagged as errors. Such an approach has also been used to review auto-contours by comparison with an independent system [13]. Where the two systems disagree, the case is flagged for editing. Such a system does not give a meaningful description of the type of error, merely reporting difference. Furthermore, both systems can agree, and both be incorrect. Therefore, this approach does not perform the same guideline-linked feedback that occurs in peer-review.

A further approach taken has been to simulate images from the contours drawn, and then compare the simulated image to the original medical image [14]. Again, differences are flagged to indicated contours that may require review. Such an approach of comparing images is further removed from the contours themselves requiring visual review of the “error maps”, and therefore human interpretable feedback on the nature of any contouring error cannot be provided to the user.

Machine learning systems have also been applied to predict a segmentation quality score [15]. However, the concept of a quality score is not present in the contouring guidelines, and no human-interpretable indication of low-quality score can be given with respect to what infringement of the guidelines may be occurring.

Therefore, it is observed that none of these systems have a method of associating the potential errors with natural language explanations. None of these systems are able to suggest changes based on the guidelines.

While such systems can highlight poor contours, they do not give the user an insight into why the contours are poor.

Therefore, while automated contour quality checking systems have been proposed. No solution adequately can be used as a form of automated “peer-review”, providing human interpretable, or natural language, feedback on where contouring guidelines have been infringed.
[1] Brouwer C L, Steenbakkers R J, van den Heuvel E, Duppen J C, Navran A, Bijl H P, Chouvalova O, Burlage F R, Meertens H, Langendijk J A, van't Veld A A. 3D variation in delineation of head and neck organs at risk. Radiation Oncology. 2012 December; 7(1):1-0.
[2] Brouwer C L, Steenbakkers R J, Bourhis J, Budach W, Grau C, Grégoire V, Van Herk M, Lee A, Maingon P, Nutting C, O'Sullivan B. CT-based delineation of organs at risk in the head and neck region: DAHANCA, EORTC, GORTEC, HKNPCSG, NCIC CTG, NCRI, NRG Oncology and TROG consensus guidelines. Radiotherapy and Oncology. 2015 Oct. 1; 117(1):83-90.
[3] https://www.nrgoncology.org/About-Us/Center-for-Innovation-in-Radiation-Oncology/Head-and-Neck/Head-and-Neck-Atlases
[4] Sun K Y, Hall W H, Mathai M, Dublin A B, Gupta V, Purdy J A, Chen A M. Validating the RTOG-endorsed brachial plexus contouring atlas: an evaluation of reproducibility among patients treated by intensity-modulated radiotherapy for head-and-neck cancer. International Journal of Radiation Oncology* Biology* Physics. 2012 Mar. 1; 82(3):1060-4.
[5] Vinod S K, Min M, Jameson M G, Holloway L C. A review of interventions to reduce inter-observer variability in volume delineation in radiation oncology. Journal of medical imaging and radiation oncology. 2016 June; 60(3):393-406.
[6] Brouwer C L, Boukerroui D, Oliveira J, Looney P, Steenbakkers R J, Langendijk J A, Both S, Gooding M J. Assessment of manual adjustment performed in clinical practice following deep learning contouring for head and neck organs at risk in radiotherapy. Physics and imaging in radiation oncology. 2020 Oct. 1; 16:54-60.
[7] The Royal College of Radiologists. Radiotherapy target volume definition and peer review RCR guidance. Clin Oncol. 2017;
[8] Rooney K P, McAleese J, Crockett C, Harney J, Eakin R L, Young V A L, et al. The impact of colleague peer review on the radiotherapy treatment planning process in the radical treatment of lung cancer. Clin Oncol. 2015; 27(9):514-8.
[9] Abrams R A, Winter K A, Regine W F, Safran H, Hoffman J P, Lustig R, et al. Failure to adhere to protocol specified radiation therapy guidelines was associated with decreased survival in RTOG 9704—A phase III trial of adjuvant chemotherapy and chemoradiotherapy for patients with resected adenocarcinoma of the pancreas. Int J Radiat Oncol Biol Phys. 2012; 82(2):809-16.
[10] Altman M B, Kavanaugh J A, Wooten H O, Green O L, DeWees T A, Gay H, Thorstad W L, Li H, Mutic S. A framework for automated contour quality assurance in radiation therapy including adaptive techniques. Physics in Medicine & Biology. 2015 Jun. 17; 60(13):5199.
[11] McIntosh C, Svistoun I, Purdie T G. Groupwise conditional random forests for automatic shape classification and contour quality assessment in radiotherapy planning. IEEE transactions on medical imaging. 2013 Mar. 6; 32(6):1043-57.
[12] Men K, Geng H, Biswas T, Liao Z, Xiao Y. Automated quality assurance of OAR contouring for lung cancer based on segmentation with deep active learning. Frontiers in Oncology. 2020 Jul. 3; 10:986.
[13] Rhee D J, Cardenas C E, Elhalawani H, McCarroll R, Zhang L, Yang J, Garden A S, Peterson C B, Beadle B M, Court L E. Automatic detection of contouring errors using convolutional neural networks. Medical physics. 2019 November; 46(11):5086-97.
[14] Brusini I, Padilla D F, Barroso J, Skoog I, Smedby Ö, Westman E, Wang C. A deep learning-based pipeline for error detection and quality control of brain MRI segmentation results. arXiv preprint arXiv:2005.13987. 2020 May 28.
[15] Chen X, Men K, Chen B, Tang Y, Zhang T, Wang S, Li Y, Dai J. CNN-based quality assurance for automatic segmentation of breast cancer in radiotherapy. Frontiers in Oncology. 2020 Apr. 28; 10:524.
[16] Feng M, Moran J M, Koelling T, Chughtai A, Chan J L, Freedman L, Hayman J A, Jagsi R, Jolly S, Larouere J, Soriano J. Development and validation of a heart atlas to study cardiac exposure to radiation following treatment for breast cancer. International Journal of Radiation Oncology* Biology* Physics. 2011 Jan. 1; 79(1):10-8.
[17] Toita T, Ohno T, Kaneyasu Y, Uno T, Yoshimura R, Kodaira T, Furutani K, Kasuya G, Ishikura S, Kamura T, Hiraoka M. A consensus-based guideline defining the clinical target volume for pelvic lymph nodes in external beam radiotherapy for uterine cervical cancer. Japanese journal of clinical oncology. 2010 May 1; 40(5):456-63.

Thus, the following problem(s) has (have) been resolved, by the present invention.

Thus, there is a need for a system and method to check contours on medical images according to contouring guidelines, and to provide feedback on the contour quality to the user. Preferably the feedback is natural language (human interpretable) feedback.

According to the invention there is provided a method for reviewing contouring of medical images in a contouring system, comprising the steps of: providing at least one medical image showing structures to be contoured; generating a contour for a structure on the at least one medical image; determining if the generated contour conforms to guidelines for the structure being contoured; in response to the determining of conformity providing feedback about the quality of the contour; and continuing to generate the contour based on the provided feedback. Preferably, the method may further comprise the steps of: repeating the determining, feedback, and contour generation steps until the structure on the medical image has been fully contoured.

In a further embodiment of the invention there is also provided a method for reviewing previously contoured images in a contouring system, comprising the steps of: loading a medical image with one of more contours; determining if at least one of the contours conforms to guidelines for the structure that has been contoured; in response to the determining of conformity with guidelines providing feedback about the quality of the contours on the medical image. Preferably, these steps are repeated until all of the contours on the medical image have been reviewed.

In a preferred embodiment of the invention, the method further comprises the step of editing the contour in response to the feedback on the quality of the contour.

Preferably, the generated contour is generated manually or semi-automatically.

In an embodiment of the invention the method further comprises the step of outputting the contour of the contoured structure of the medical image. Further preferably, the steps of the method are repeated so that multiple structures in the at least one medical image are contoured.

In a preferred embodiment of the invention, the method further comprising the step of: displaying one or more contours on the medical image before the user starts to generate a manual contour for the medical image. Preferably, the method further comprises the step of: displaying one or more contours on the medical image before the system determines if the one or more contours conforms to the guidelines.

Preferably, the method also comprising the step of determining at least one infringement zone from the guidelines, wherein the at least one infringement zone is used to determine a region of the scan image where a contour for the structure being contoured should not be placed. Preferably, the at least one infringement zone is determined using Machine Learning on example medical images. In a further embodiment of the invention, the method comprises the step of providing additional spatial feedback about the position of the contour relative to the position of the at least one infringement zone. Preferably, the additional spatial feedback for each of the at least one infringement zones, is specific to the image area covered by the at least one infringement zone. In a preferred embodiment of the invention the additional spatial feedback is provided by highlighting an area on the image where the contour infringes the infringement zone. In a yet further embodiment of the invention, the additional spatial feedback is provided as soon as the infringement of the infringement zone by the contour occurs.

Further preferably, the guidelines for contouring refer to one or more of: anatomical boundaries between structures; other anatomical structures; other anatomical features.

In an embodiment of the invention a reference anatomy on the medical image is identified using at least one of; atlas-based auto-contouring; machine learning methods; algorithmic approaches.

Preferably, a comparison of the relative position of the contour to the reference anatomy in the medical image is used to determine adherence to guidelines. Further preferably, a tolerance is applied to the contour with respect to the reference anatomy. In an embodiment of the invention, the tolerance is determined using at least one of isotropic or directional margins.

In an embodiment of the invention the at least one medical image is one of a 2D image, a 3D image or a time series of medical images. Further preferably, the at least one medical image is a CT scan, CBCT scan, PET scan, SPECT scan or an MRI scan.

In an embodiment of the invention, the feedback is natural language or human interpretable feedback. Preferably the feedback is provided as a report. In a further embodiment of the invention the feedback is provided by annotation of the contours to indicate the type of error with the contour. In an embodiment of the invention the annotation is one or more of: symbolic annotations, textual annotations linked to the line; line weighting change, line colour change, line style change, shading/colouring around the line; shading colouring within the infringing portion of the contour

Preferably, a user can select the appropriate guidelines according to the structure being contoured or the contoured structure in a pre-contoured image.

Further preferably, the guidelines to be provided are automatically determined according to the structure being contoured.

Preferably, the method of determining if the generated contour conforms to guidelines is derived from a database of previously contoured images. Further preferably, the database includes previous feedback on whether/how the previous contours varied from the guidelines.

According to the invention there is also provided a system for contouring of at least one medical image comprising: a display for displaying at least one medical image to be contoured by a user; a processor for determining that the user has initiated contouring of a structure on the medical image; the processor determining if the generated contour conforms to guidelines for the structure that is being contoured; in response to the determination of contour conformity with the guidelines, providing feedback to the user about the quality of the contour; so that the user can adjust the contour to take account of the provided feedback.

Preferably, the generated contour is generated manually or semi-automatically.

In a further embodiment of the invention, there is provided a system for analysing a medical image with contoured structures comprising: an input for receiving at least one contoured medical image; a processor for determining if at least one of the contours conforms to guidelines for the structure that has been contoured; in response to the determining of conformity with guidelines the processor providing feedback about the quality of the contours on the medical image.

Preferably, the system further comprises a display for displaying the at least one contoured image.

### BRIEF DESCRIPTION OF THE DRAWINGS

Further details, aspects and embodiments of the invention will be described, by way of example only, with reference to the drawings. In the drawings, like reference numbers are used to identify like or functionally similar elements. Elements in the figures are illustrated for simplicity and clarity and have not necessarily been drawn to scale.

FIG. 1 is a flow chart showing the method according to an embodiment of the invention;

FIG. 2 is a flow chart showing the method according to an alternative embodiment of the invention;

FIG. 3a shows a medical scan image with a first contour;

FIG. 3b shows a medical scan image with an infringement zone;

FIG. 3c shows a medical scan image showing potential regions of error;

FIG. 4: shows a medical scan image with a contour that does not meet contouring guidelines;

FIG. 5: shows a medical scan image alongside a report to provide feedback on the image contours;

FIG. 6: illustrates a simplified block diagram of an example of a medical imaging system.

### DETAILED DESCRIPTION

The disclosed invention is a system and method that can offer guideline related feedback for contours on a medical image. The feedback may be provided for review of existing contours, or for contours that are newly generated on a medical image. The feedback may be related to the quality of the contour. Additional feedback may also be provided including, for example feedback on the spatial properties of the contour, such as the position of the contour on the medical image. Preferable the feedback is natural language feedback or some other human interpretable feedback. In some cases, the feedback may be visual or audio feedback. Alternatively, the feedback may be provided as a report. In an embodiment of the invention the system and method may provide feedback through an interface while the user draws or edits contours, or retrospectively once the user has finished drawing the contours, depending on user preference. In an embodiment of the invention the contours are generated either manually or semi-automatically. Preferably, the interface will be a visual interface that is provided to the user as part of the system. The system and method identify structures of interest in medical scans and verifies whether the contours for the structures of interest adhere to the guidelines specific to each structure. In a preferred embodiment of the invention the guidelines for contouring refer to one or more of: anatomical boundaries between structures; other anatomical structures; other anatomical features. Preferably, a user can select the appropriate guidelines according to the structure being contoured or the contoured structure in a pre-contoured image. In an embodiment of the invention, the guidelines to be provided are automatically determined according to the structure being contoured.

Preferably the medical image to be contoured, or for contour review is a CT scan, but other scans such as MRI, CBCT, PET, or SPECT may also be used in other embodiments of the invention. In an embodiment of the invention the at least one medical image is one of a 2D image, a 3D image or a time series of medical images. Where it is determined that a contoured structure does not adhere to the appropriate guideline for the structure, the user is informed of the nature of the infringement. The system and method determine what guidelines are appropriate for the body region and structures of interest, evaluates the contours on the medical scan against the guidelines and offers appropriate feedback, preferably natural language or human interpretable feedback on potential infringements of the guidelines. In an embodiment of the invention, the system and method may indicate to the user the nature of the potential contouring error in a range of ways, including but not limited to one or more of; reports, screenshots, annotations of contours, highlighting of contours with colour, fill, or shading, indicating regions through colour, fill or shading, on-screen text annotations of the contour, or other parts of the image, tool-tips on contours, audio feedback from the system, indication of the location or type of infringement on an anatomic diagram, highlighting of relevant paragraphs of guidelines, preferably automatic highlighting, display of relevant guideline rules. The feedback may link multiple forms of feedback, for example a report may refer to a screenshot.

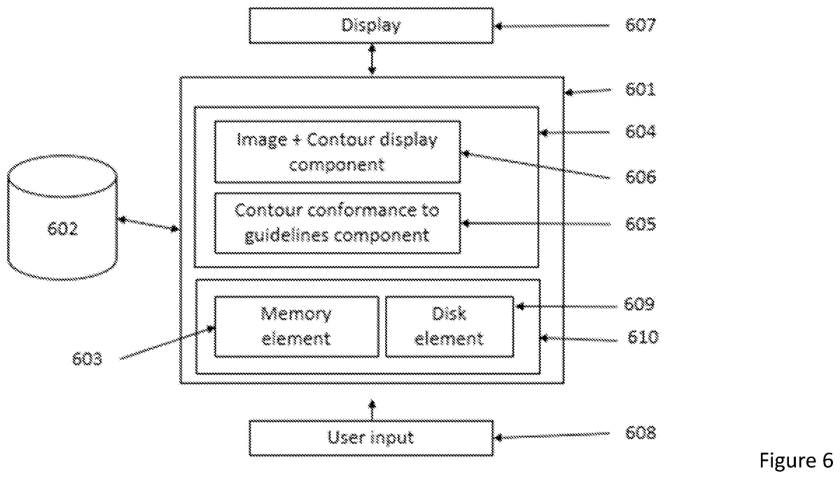

Referring now to FIG. 6, there is illustrated a simplified block diagram of an example of a medical imaging system 600 arranged to enable medical images to be displayed to a user to be used in the method of this invention, for contouring, or review of existing contours. Preferably the system comprises at least a display for displaying at least one medical image to be contoured by a user; and a processor for determining that the user has initiated contouring of a structure, or contour review of an existing contoured structure on the medical image. Preferably, the processor determines if the generated contour conforms to guidelines for the structure that is being contoured; in response to the determination of contour conformity with the guidelines, and provides feedback to the user about the quality of the contour; so that the user can adjust the contour to take account of the provided feedback. Alternatively, the processor may determine if at least one of the contours on a structure with existing contours conforms to guidelines for the structure that has been contoured; and in response to the determining of conformity with guidelines the processor provide feedback about the quality of the contours on the medical image.

In the illustrated example, the medical imaging system 600 comprises one or more user terminals 601, for example, comprising a workstation or the like, arranged to access medical images stored within, for example, a database 602 or other data storage apparatus. In an embodiment of the invention, the system also comprises an input for receiving at least one contoured medical image, or image to be contoured. In the illustrated example, a single database 602 is illustrated. However, it will be appreciated that the user terminal 601 may be arranged to access medical images from more than one data storage apparatus. Furthermore, in the illustrated example the database 602 is illustrated as being external to the user terminal 601. However, it will be appreciated that the user terminal 601 may equally be arranged to access medical images stored locally within a local storage module illustrated at 610 on one or more internal storage elements, such as the memory element illustrated at 603 or the disk element illustrated at 609. The user terminal 601 further comprises one or more signal processing modules, such as the signal processing module illustrated generally at 604. The signal processing module(s) is/are arranged to executing computer program code, for example stored within local storage module 609. In the illustrated example, the signal processing module(s) 605 is/are arranged to execute computer program code comprising one or more of the automated contour quality checking component(s) illustrates in the example as the contour conformance to contouring guidelines component 605. The signal processing module 604 in the illustrated example is further arranged to execute computer program code comprising one or more image display component(s) 606; the image display component(s) 606 being arranged to display the images with the generated or loaded contours and the feedback on the quality of the contours such the one generated by the contour conformance to contouring guidelines component(s) 605, to a user, for example on a display screen 607 or the like. The medical imaging system 600 may further comprise one or more user input devices, such as illustrated generally at 608, to enable a user to interact with computer program code etc. executing on the signal processing module(s) 604.

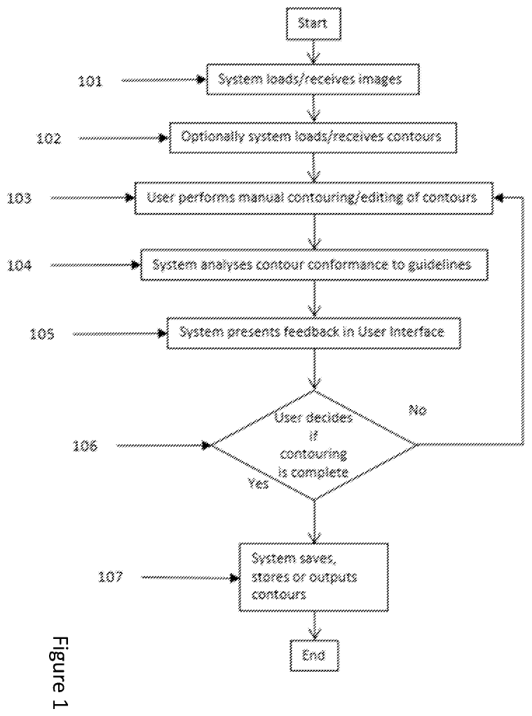

One embodiment of the method of the invention is illustrated by FIG. 1. The system loads at least one medical image at step 101. The medical image may be 2D image or a 3D image, or a time-series of medical images, where the time series of medical images are a sequence of images of the same anatomy acquired in the same scanning session—i.e. with the patient remaining in the scanner throughout, for example, a 4D image usually comprise of a series of 3D images, where each 3D image represents a different phase of a respiratory or cardiac cycle (a specific time point in a time series). In this embodiment of the invention the medical image is a CT image, but other medical images may be used as previously described. This image loading process may have been user initiated or may have been automatic. Optionally, the system may also load one or more existing contours at step 102, for review and/or editing by the user, should this be required after the review. Preferably, the editing of the contours is interactive (manual editing or semi-automatic editing). The loaded/received contours can be generated by any contouring method. The editing/review of the contours may be for all ROIs (regions of interest) on the image, or just some of the ROIs, depending on the expertise of the user.

It is anticipated that such a system for contour reviewing has normal viewing capabilities of the medical image visualisation tool, and the user may opt to change the view. That is, the system has the ability to visualise the image (with contours) using different layouts of a combination of any of the views (axial, sagittal, coronal, possibly 3D rendering). Furthermore, such a system is likely to have the ability to window/level the image, change contrast, navigate within the 3D image, zoom, pan. In an embodiment of the invention, one or more contours are displayed on the medical image before the user starts to generate a manual contour for the medical image. At some point the user initiates the contouring process, to generate a contour for the loaded medical image at step 103, using manual or semi-automatic contouring tools available in the system. Such tools typically comprise click-based polygon drawing tools, pen-like freehand drawing tools, and/or brush-like region filling tools. The system then determines if the contour that has been generated or loaded onto the image conforms to the contouring guidelines for the structure being contoured, at step 104, preferably this is done automatically by the system. Where it is determined that the generated contour, or part of the generated contour infringes the guidelines for the structure being contoured, the system presents this information as feedback on the guideline infringement to the user in the user interface, preferably in a human interpretable format, at step 105. In a preferred embodiment of the invention the method of determining if the generated contour conforms to guidelines is derived from a database of previously contoured images. Preferably, the database includes previous feedback on whether/how the previous contours varied from the guidelines. In an embodiment of the invention, feedback about the quality of the generated contour is provided in response to the determining the conformity to the guidelines. Preferably, the user then continues to generate the contour based on the provided feedback. The user then may also continue contouring/editing one or more contours by repeating these steps until they are satisfied, taking account of the feedback, to ensure the best possible contour or contours are generated, and the structure on the medical image as been fully contoured. On completing contouring of the medical image at step 106, the system saves, stores or outputs the contours at step 107. Output of the contour of the contoured structure of the medical image would normally be in a format commonly used within the medical device community, such as DICOM format. DICOM is also an image transfer protocol, thus the data may be transferred to another devices such as a Treatment Planning System (TPS) or a Picture Archiving and Communications System (PACS). Alternatively, the contours may be stored to a local or network file system. In a preferred embodiment of the invention the steps in FIG. 1 may be repeated so that one or more structures in the at least one medical image have been contoured.

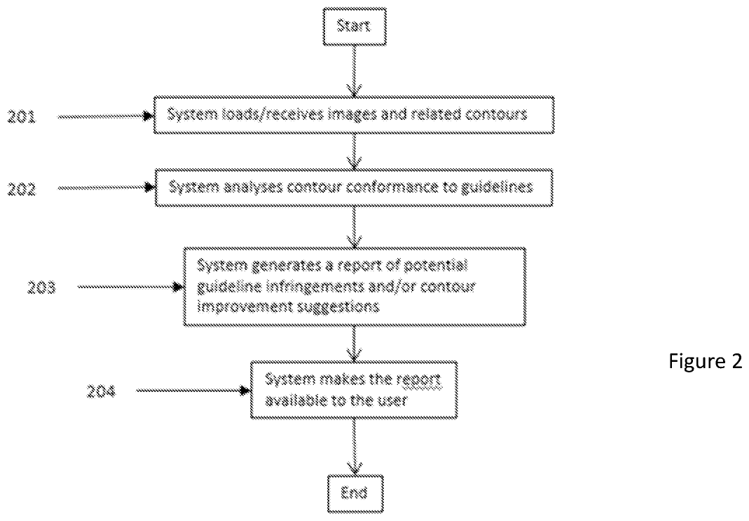

An alternative embodiment of the invention operating is illustrated by FIG. 2. This embodiment relates to review of existing contours on previously contoured images for conformity with the appropriate guidelines, rather than the generation of new contours on a medical image. The system loads at least one medical image (which may be 2D or 3D, or a time-series of medical images) and a related set of one of more contours, at step 201. This image loading process may have been user initiated or may have been automatic. In an embodiment of the invention the method may also include the step of displaying one or more contours on the medical image before the system determines if the one or more contours conforms to the guidelines. The system then determines if the one or more contours for review conform to the guidelines for the structure that has been contoured, at step 202, preferably this is an automatic determination. In response to the determination of conformity to the guidelines the system provides feedback about the quality of the contours on the medical image. The system may also generate a report detailing potential infringement of one or more guidelines by the reviewed contour. Preferably, the system will also suggest possible improvements that could be made to the contours, based on the provided feedback. Preferably the improvements are suggested in a natural language or human interpretable form, at step 203. The system may also make the report available to the user at step 204. In an embodiment of the invention the report can be exported/stored with any document format (PDF, DOC etc) including DICOM. Alternatively, the report may be either directly/immediately displayed on the screen or stored (in DICOM or any other format) to be retrieved later, or the report could be emailed or made available as a download, or even sent directly to a printer if a hard copy is required. In an embodiment of the invention, the method may also comprise the step of editing the contour in response to the feedback on the quality of the contour. In a preferred embodiment of the invention the steps in FIG. 2 may be repeated until all of the contours on a medical image have been reviewed.

In some embodiments of the invention the system may also provide feedback based on experience learnt from peer review (preferably manual peer review) of contours or using one or more examples of erroneous contours. Machine learning methods may be used to learn the correspondence between one or more contours, their location on the medical image, and the prior human interpretable explanations/knowledge of how the contours infringe guidelines or may be improved. To do so, a database of previous contours is required, together with information on how the contours infringe guidelines or how the contours may be improved. Optionally, this database of previous contours would also include the medical image of the patient, such that the relationship between a contour and image can be learnt. A machine learning system is then trained with this database of contours and the optional medical image, to develop a model which can predict, for a given contour and optionally the associated patient medical image, what feedback would be appropriate to provide. Such an approach may be trained per body region, or guideline, or for several body regions and/or guidelines. Once a model is developed, the system then uses the trained model to analyse user contours (steps 104 in FIG. 1, and step 202 in FIG. 2). Since the system has been trained with examples of human interpretable feedback, the system is able to provide the feedback on the user's contour(s) in a human interpretable form. For example, the feedback may be provided as information displayed on a screen, annotations of the contours or some other means.

In some embodiments of the invention, “infringement zones” are defined on example medical images with additional linked spatial feedback. Typically an image will have at least one infringement zone. These infringement zones are areas of the medical image where a contour for a structure being contoured should not be placed. For example, when a guideline states “Superiorly, the whole heart starts just inferior to the left pulmonary artery”, a region superior to the left pulmonary artery can be identified and automatically annotated on example medical images as an “infringement zone”, where the contour for the heart should not be placed. This infringement zone may also be linked with the feedback “Guidelines state that the heart should start inferior to the pulmonary artery. The drawn contour appears to extend too far superiorly”. This feedback could be provided by display on the screen, as an audio message to the user. These one or more infringement zones are then mapped onto or detected/predicted on the patient medical image that is being contoured or evaluated by the user. In a preferred embodiment of the invention, the infringement zones may not be visible to the user. Preferably, the regions of the image where the contour infringes the infringement zone may be clearly identified to the users. In an embodiment of the invention the method further comprised the step of determining at least one infringement zone from the guidelines, wherein the at least one infringement zone is used to determine a region of the scan image where a contour for the structure being contoured should not be placed. Preferably, the at least one infringement zone is determined using Machine learning on example medical images, The area of the contour that has infringed the infringement zone will be clearly identifiable based on the colour, shading, or line weighing of the boundary of the contour. Preferably this mapping of the infringement zones may be performed using image registration to find the mapping between the patient medical image and the example medical images. Typically, image registration finds the mapping between the patient image and the example medical image by the transformation, whether locally or globally, that maximizes a similarity measure between the two images after the transformation has been applied. The use of multiple local transformations at different points in the image, is referred to as deformable registration. A global transformation for the image is referred to as a rigid or affine registration. In a further embodiment of the invention infringement zones may also be learnt from example medical images using machine learning methods to generate a machine learning model. This machine learning model is then applied to the patient medical image to predict the infringement zones on the patient medical image for the structure or region of interest to be contoured. Other approaches to identify regions of interest on medical images, such as classical algorithmic approaches, including but not limited to thresholding, a watershed method, a level-set method, and the graph-cut method, will be known to those skilled in the art [18]. On analysing the patient medical image (steps 104 in FIG. 1, and step 202 in FIG. 2), the system compares the user generated contour to the infringement zone for the contour, as provided from the guidelines. The system is then able to provide specific spatial feedback about the position and other spatial parameters of the contour relative to the position of the at least one infringement zone. Where the user generated contour overlaps with an infringement zone, the additional linked spatial feedback is then be provided to the user, either visually, or using audio feedback, or some other feedback system. In an embodiment of the invention the spatial feedback is feedback about the position of the contour relative to the position of the at least one infringement zone. Further preferably, the additional spatial feedback for each of the at least one infringement zones, is specific to the image area covered by the at least one infringement zone. Preferably the spatial feedback is provided instantly, as soon as the infringement of the infringement zone by the contour occurs. That is, as soon as the generated contour meets the infringement zone while the user is performing contouring or editing. However, in some cases, the system may wait until a contour is completed before providing the spatial feedback of possible overlap of the contour with the infringement zone. In an embodiment of the invention the additional spatial feedback is provided by highlighting an area on the image where the contour infringes the infringement zone. The system may also highlight the potentially erroneous part of the contour to the user, by highlighting this in some way on the contoured image or informing the user in some other way. In some embodiments of the invention the assessment of contours may be performed retrospectively on contours that have been previously generated. In such an embodiment the feedback can be reported in a user interface, with the images and contoured displayed to the users. In an alternative embodiment the retrospective assessment of the contours could occur automatically within the system, with the feedback provided to the user as report, sent by example by email or downloadable from a webpage.

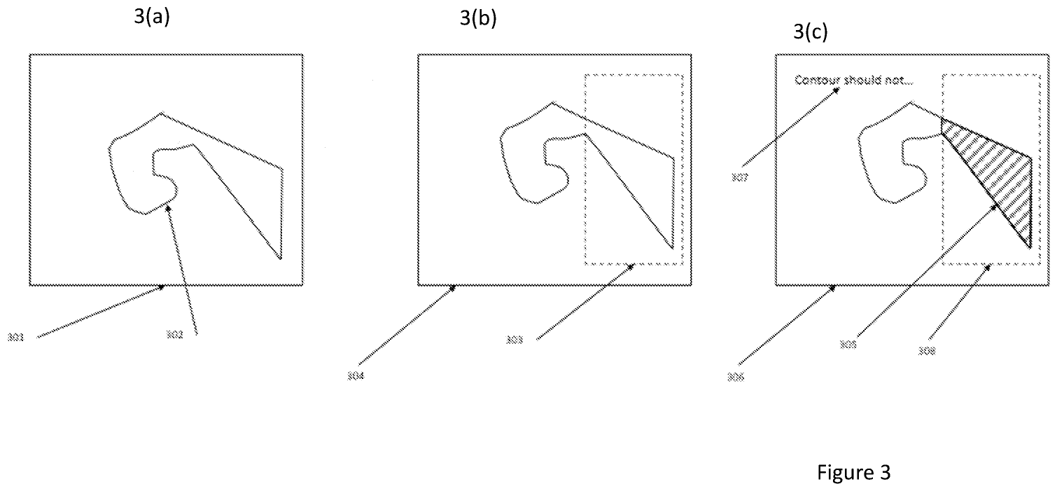

Such an embodiment of the invention is illustrated in FIG. 3. FIG. 3a shows a patient medical image, 301, on which the user has drawn a contour 302. This will typically be drawn either manually or semi-automatically. In FIG. 3b, an automatic “infringement zone”, 303, has been mapped onto the patient medical image, 304, by the system. Note 301 and 304 represent the same patient image at different stages of the process. In FIG. 3c, the system indicates the regions of potential error to the users graphically, 305, on the patient image, 306, and provides the explanation of the potential guideline infringement, 307, that was linked to the infringement region 308. As shown, this indication and explanation is provided visually, but it may be provided in different ways, for example as audio feedback. Note, 308 represents the same as 303 and 306 represents the same image as 304, but at a different stage of the process. As described above, the infringement zone is not necessarily displayed to the user, preferable it is not shown at all. In a preferred embodiment of the invention the region where the contour infringes the infringement zone may be highlighted using a different colour, annotations, line thickness or line style to the portion of the contour in the infringement zone, or by shading or hashing the area within or around the portion of the contour within the infringement zone.

In another embodiment of the invention, the system and method is configured to apply one or more rules to detect possible guideline infringement for the generated contour. As noted in the background, many guidelines use relationships to reference anatomy to define the target structure. In this embodiment of the invention the system automatically determines the location of reference anatomy. Preferably, a comparison of the relative position of the contour to the reference anatomy in the medical image is used to determine adherence to guidelines. The system applies one or more rules derived from the guidelines for contouring. These rules may be pre-configured in the system, loaded from a database, configured by the user, or determined using natural language processing of the guidelines. The system identifies whether one or more of the rules have been infringed. On determining a rule has been infringed, the system reports the infringement back to the user in a human interpretable format, such as text display, highlighting a problematic region of a contour with a different colour, shading, or weight etc.

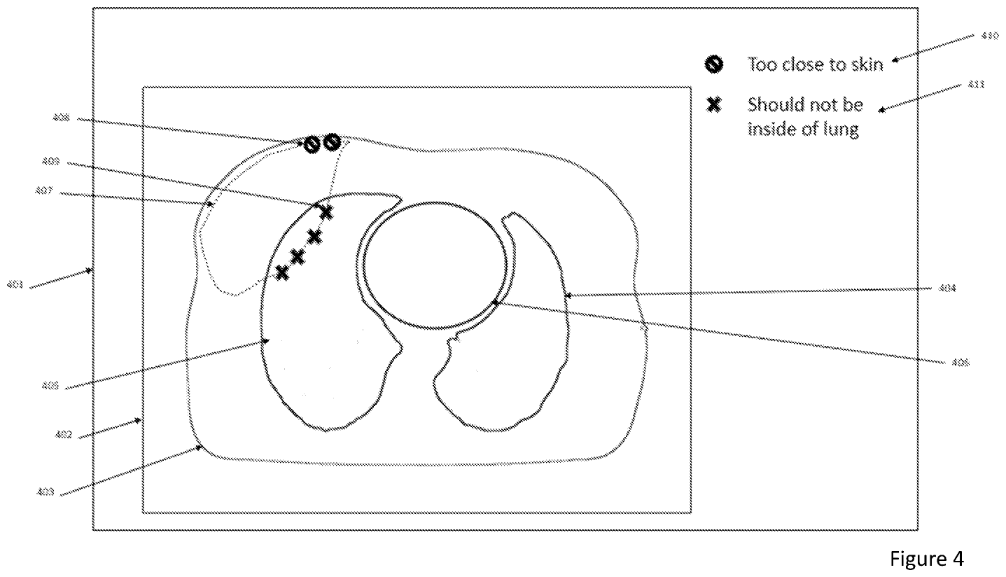

Such an embodiment of the invention is illustrated in FIG. 4. The system is showing a user interface, 401, on a display screen such as computer monitor or tablet device for example or any other display device suitable for use for contouring. Within that user interface is displayed a patient medical image, 402, preferably loaded in response to a user action. Preferably the image is a CT image, but other imaging techniques may be used to provide the medical image being contoured. The medical image shows the cross-section of a patient with the patient skin outline, 403, the left lung, 404, the right lung, 405, and the heart, 406, illustrated. The user has drawn a contour, 407, for the right breast. The contour is drawn manually or semi-automatically. The system has identified the reference anatomy of the patient skin outline, 403, the left lung, 404, the right lung, 405, and the heart, 406. Preferably, this identification is done automatically by the system. The system has applied one or more rules from the guidelines that the contour 407 for the right breast should not overlap the lung tissue, and the breast contour 407 should be 5 mm from the skin boundary. The system provides this feedback in human interpretable form, preferably through the use of annotations on the contours 408 and 409, together with a key to provide interpretation of the annotation, 410 and 411, within the user interface. The key may be provided on the screen, or in a separate look-up table or alternatively as an audio output, alternatively the key may be provided separately to the user interface, for example in a user manual. In a preferred embodiment of the invention the annotation to the contour may be one or more of: symbolic annotations, textual annotations linked to the line, line weighting change, line colour change, line style change, shading/colouring around the line; shading colouring within the infringing portion of the contour.

Methods for detecting the reference anatomy will include methods known to those skilled in the art [18] and could include (but not limited to) atlas-based contouring, image registration from an example reference, machine learning based contour identification, or classical algorithmic methods, including but not limited to thresholding, the watershed, the level-set and the graph-cut methods.

The system could apply one or more rules including, but not limited to, that apply a relative location to reference anatomy (i.e, must be superior/inferior/left/right/anterior/posterior to . . . ), check for inclusion or exclusion of the reference anatomy within the contour, perform distance measurements to the reference anatomy, apply a tolerance to the contour with respect to reference anatomy, by for example adding isotropic or directional margins. Further rules may consider the structure itself, without comparison to a reference anatomy, for example “the target structure should be a single contiguous volume” or “the target structure should not contain holes”.

The system may indicate to the user the nature of the potential contouring error in a range of ways, including but not limited to one or more of; reports, screenshots, annotations of contours, highlighting of contours with colour, fill, or shading, indicating regions through colour, fill or shading, on-screen text annotations, tool-tips on contours, audio feedback from the system, indication of the location or type of infringement on an anatomic diagram, automatic highlighting of relevant paragraphs of guidelines, display of relevant guideline rules. The feedback may link multiple forms of feedback, for example a report may refer to a screenshot.

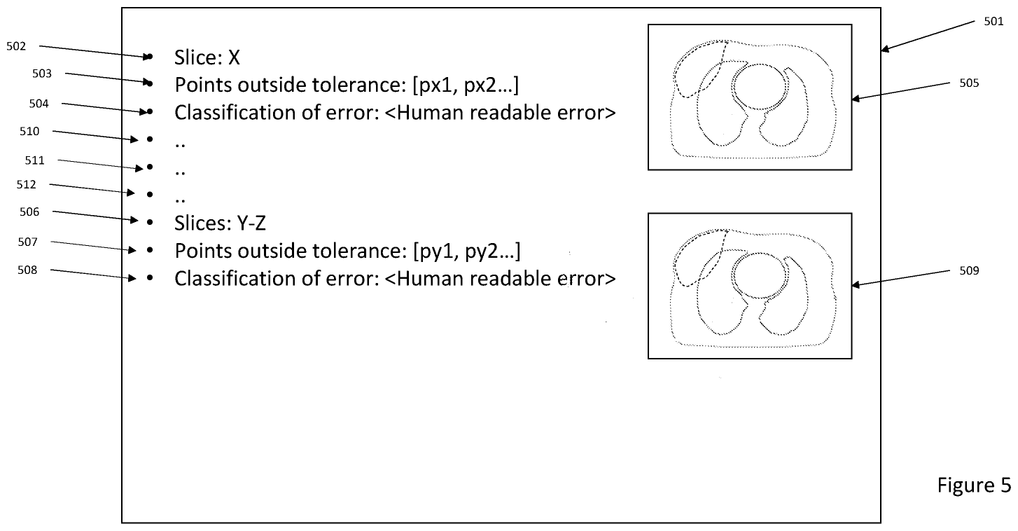

FIG. 5 illustrates the use of a report to provide feedback to the user. In this embodiment of the invention, the report and images are displayed on a user display, but as described previously the report may also be provided in any document format (PDF, DOC etc) including DICOM. Alternatively, the report may be either directly/immediately displayed on the screen or stored (DICOM or any other format) to be retrieved later, or the report could be emailed or made available as a download, or even sent directly to a printer if a hard copy is required. The report has been generated by the system and is made available to the user. The report is illustrated in 501. A single error is encapsulated in 502, 503, 504, together with screenshot, 505. Another error is encapsulated in 506, 507, 508, and screenshot, 509. In an embodiment of the invention, the <Human readable error>, identified at 504 and 508, could take the form of a statement like “The BREAST contour is too close to the SKIN boundary. The guidelines state the contour should be 5 mm from the skin” or “The BREAST contour overlaps the LUNG tissue”. The additional bullets points shown at 510, 511 and 512 are used to illustrate that there may be any number of potential errors identified, with at least one potential error being reported. The slice, 502, or slices, 506, related to the potential error detected are shown.

The geometric location or locations representing the contour that may be in error are shown 503 and 507. A human interpretable classification of the error is included in the report, 504 and 508. Such a report may be human readable directly or may be stored in machine readable format to allow the report to be visualised by the user textually or graphically in a user-interface. The report may also be saved for later analysis and to help with updating guidelines or other work.

The system preferably applies the appropriate one or more guidelines to a contour. There are various ways that this can be implemented to enable the correct one or more guidelines to be applied; The user can select the appropriate one or more guidelines to apply, this could be for example via a menu item, dropdown selection, or button, in the user-interface, or by launching the system with a specific configuration. The system may be configured to detect which one or more guidelines to apply based on the naming of the structure or structures contoured, detection of the region of the anatomy, or by comparison to example template images and contour sets (known as atlases). Preferably this may be automatic detection by the system. The detection of the region of the anatomy can be performed using machine learning models trained to classify images into relevant anatomy using prior examples. Comparison to example template images and contour sets, known as atlases, can be performed using deformable image registration to map contours between the patient image and the atlas image. This process is well known to those skilled in the art [19]. The distance or overlap between contours can be used to determine the quality of match and therefore if the anatomy is the same. For example, the atlas may contain regions for all of the expected structures to be contoured. These are mapped to the patient image using deformable image registration. These contours are not shown to the user, rather the user's contour is compared with each of the atlas contours. The region of anatomy the user is contouring can be determined by finding the atlas region with which there is a greatest percentage overlap, or by determining the region where the average distance from the user contour to the nearest point on the mapped atlas contour is lowest. Intensity-based image similarity measures, such as mutual information, correlation coefficient or sum-of-squared-differences, can be used to assess the quality of match between the atlas image and the patient image to determine the region of anatomy, where the atlas images available represent different regions of anatomy. Such similarity measures are well-known to those skilled in the art [20]. Different atlases for the different anatomical regions can be used to determine the most similar anatomical region. For instance, if two atlases were available, representing the pelvis and the thoracic region respectively, the sum-of-squared differences following deformable image registration would be lower to the thoracic atlas than the pelvic atlas if the patient case is a thoracic image. Thus, it can be determined that the user is contouring in the thoracic region and therefore thoracic contouring guidelines must be applied as a reference because the sum-of-squared difference similarity measure is lower. Similar approaches can be taken with other similarity measures accounting for whether the similarity measure is maximized or minimized for similar images. The reference guidelines for contouring or contour reviewing may be determined from look-up table, database or other storage, based on the target structure being assessed by the user. In an embodiment of the invention, a user may select the appropriate guidelines according to the structure being contoured or the contoured structure in a pre-contoured image. Alternatively, the guidelines to be provided may be automatically determined according to the structure being contoured. The user could choose to assess a single target structure, or multiple target structures within an image.
[18] Sharp G, Fritscher K D, Pekar V, Peroni M, Shusharina N, Vecraraghavan H, Yang J. Vision 20/20: perspectives on automated image segmentation for radiotherapy. Medical physics. 2014 May; 41(5):050902.
[19] Rohlfing T, Brandt R, Menzel R, Russakoff D B, Maurer C R. Quo vadis, atlas-based segmentation?. InHandbook of biomedical image analysis 2005 (pp. 435-486). Springer, Boston, MA.
[20] Penney G P, Weese J, Little J A, Desmedt P, Hill D L. A comparison of similarity measures for use in 2-D-3-D medical image registration. IEEE transactions on medical imaging. 1998 August; 17(4):586-95.

Examples of this invention may be applied to any or all of the following: Picture archiving and communication systems (PACS); Advanced visualisation workstations; Imaging Acquisition Workstations; Web-based or cloud-based medical information and image systems; Radiotherapy Treatment planning system (TPS); Radiotherapy linear accelerator consoles; Radiotherapy proton beam console.

The present invention has been described with reference to the accompanying drawings. However, it will be appreciated that the present invention is not limited to the specific examples herein described and as illustrated in the accompanying drawings. Furthermore, because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

The invention may be implemented in a computer program for running on a computer system, at least including code portions for performing steps of a method according to the invention when run on a programmable apparatus, such as a computer system or enabling a programmable apparatus to perform functions of a device or system according to the invention.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system. Therefore, some examples describe a non-transitory computer program product having executable program code stored therein for automated contouring of cone-beam CT images.

The computer program may be stored internally on a tangible and non-transitory computer readable storage medium or transmitted to the computer system via a computer readable transmission medium. All or some of the computer program may be provided on computer readable media permanently, removably or remotely coupled to an information processing system. The tangible and non-transitory computer readable media may include, for example and without limitation, any number of the following: magnetic storage media including disk and tape storage media; optical storage media such as compact disk media (e.g., CD-ROM, CD-R, etc.) and digital video disk storage media; non-volatile memory storage media including semiconductor-based memory units such as FLASH memory, EEPROM, EPROM, ROM; ferromagnetic digital memories; MRAM; volatile storage media including registers, buffers or caches, main memory, RAM, etc.

A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input, and responds by allocating and managing tasks and internal system resources as a service to users and programs of the system.

The computer system may for instance include at least one processing unit, associated memory and a number of input/output (I/O) devices. When executing the computer program, the computer system processes information according to the computer program and produces resultant output information via I/O devices.

In the foregoing specification, the invention has been described with reference to specific examples of embodiments of the invention. It will, however, be evident that various modifications and changes may be made therein without departing from the scope of the invention as set forth in the appended claims and that the claims are not limited to the specific examples described above.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures can be implemented which achieve the same functionality.

Any arrangement of components to achieve the same functionality is effectively ‘associated’ such that the desired functionality is achieved. Hence, any two components herein combined to achieve a particular functionality can be seen as ‘associated with’ each other such that the desired functionality is achieved, irrespective of architectures or intermediary components. Likewise, any two components so associated can also be viewed as being ‘operably connected,’ or ‘operably coupled,’ to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms ‘a’ or ‘an,’ as used herein, are defined as one or more than one. Also, the use of introductory phrases such as ‘at least one’ and ‘one or more’ in the claims should not be construed to imply that the introduction of another claim element by the indefinite articles ‘a’ or ‘an’ limits any particular claim containing such introduced claim element to inventions containing only one such element, even when the same claim includes the introductory phrases ‘one or more’ or ‘at least one’ and indefinite articles such as ‘a’ or ‘an.’ The same holds true for the use of definite articles. Unless stated otherwise, terms such as ‘first’ and ‘second’ are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

1-35 (canceled)

36. A method for reviewing contouring of medical images in a contouring system, comprising the steps of:
providing at least one medical image showing structures to be contoured;
generating a contour for a structure on the at least one medical image;
automatically determining if the generated contour conforms to guidelines specific to the structure being contoured;
in response to the determining of conformity providing feedback about the quality of the contour; and
continuing to generate the contour based on the provided feedback.

37. A method for reviewing previously contoured images in a contouring system, comprising the steps of:
loading a medical image with one of more contours;
automatically determining if at least one of the contours conforms to guidelines specific to the structure that has been contoured;
in response to the determining of conformity with guidelines providing feedback about the quality of the contours on the medical image.

38. A method as claimed in claim 37, further comprising the step of editing the contour in response to the feedback on the quality of the contour.

39. A method as claimed in claim 36, further comprising the steps of repeating the determining, feedback, and contour generation steps until the structure on the medical image has been fully contoured.

40. A method as claimed in claim 36, wherein the generated contour is generated manually or semi-automatically.

41. A method as claimed in claim 36, further comprising the step of outputting the contour of the contoured structure of the medical image.

42. A method as claimed in claim 36, further comprising the step of:
displaying one or more contours on the medical image before the user starts to generate a manual contour for the medical image.

43. A method as claimed in claim 38, further comprising the step of:
displaying one or more contours on the medical image before the system determines if the one or more contours conforms to the guidelines.

44. A method as claimed in claim 36, further comprising the step of determining at least one infringement zone from the guidelines, wherein the at least one infringement zone is used to determine a region of the scan image where a contour for the structure being contoured should not be placed.

45. A method as claimed in claim 44, wherein the at least one infringement zone is determined using Machine Learning on example medical images.

46. A method as claimed in claim 44, further comprising the step of providing additional spatial feedback about the position of the contour relative to the position of the at least one infringement zone; wherein the additional spatial feedback for each of the at least one infringement zones, is specific to the image area covered by the at least one infringement zone and the additional spatial feedback is provided by highlighting an area on the image where the contour infringes the infringement zone.

47. A method as claimed in claim 36, wherein the guidelines for contouring refer to one or more of: anatomical boundaries between structures; other anatomical structures; other anatomical features.

48. A method as claimed in claim 47, wherein a reference anatomy on the medical image is identified using at least one of;
atlas-based auto-contouring; machine learning methods; algorithmic approaches; wherein a comparison of the relative position of the contour to the reference anatomy in the medical image is used to determine adherence to guidelines.

49. A method as claimed in claim 36, wherein the at least one medical image is at least one of: a CT scan CBCT scan, PET scan, SPECT scan or an MRI scan.

50. A method as claimed in claim 36, wherein the feedback is natural language or human interpretable feedback, and is provided as one of more of: a report, by annotation of the contours to indicate the type of error with the contour; wherein the annotation is one or more of: symbolic annotations, textual annotations linked to the line; line weighting change, line colour change, line style change, shading/colouring around the line; shading colouring within the infringing portion of the contour.

51. A method as claimed in claim 36, wherein a user can select the appropriate guidelines according to the structure being contoured or the contoured structure in a pre-contoured image.

52. A method as claimed in claim 36, wherein the guidelines to be provided are automatically determined according to the structure being contoured.

53. A method as claimed in claim 36, wherein the method of determining if the generated contour conforms to guidelines is derived from a database of previously contoured images; wherein the database includes previous feedback on whether/how the previous contours varied from the guidelines.

54. A system for contouring of at least one medical image comprising:
a display for displaying at least one medical image to be contoured by a user;
a processor for determining that the user has initiated contouring of a structure on the medical image;
the processor determining if the generated contour conforms to guidelines specific to the structure that is being contoured; in response to the determination of contour conformity with the guidelines, providing feedback to the user about the quality of the contour; so that the user can adjust the contour to take account of the provided feedback.

55. A system as claimed in claim 54, wherein the generated contour is generated manually or semi-automatically.
